(19) United States
(12) Patent Application Publication
(10) Pub. No.: US 2019/0307696 A1
(43) Pub. Date: Oct. 10, 2019

(54) TABLETS COMPRISING MIRABEGRON AND SOLIFENACIN

(71) Applicant: SYNTHON B.V., Nijmegen (NL)

(72) Inventors: Agnes FERNANDEZ PENA, Sant Boi de Llobregat (ES); Jose VELADA CALZADA, Nijmegen (NL); Rohit KUMAR, Sant Boi de Llobregat (ES); Lisardo ALVAREZ FERNANDEZ, Sant Boi de Llobregat (ES)

(21) Appl. No.: 16/096,499

(22) PCT Filed: Apr. 21, 2017
(86) PCT No.: PCT/EP2017/059546

§ 371 Date: Oct. 25, 2018

(30) Foreign Application Priority Data
Apr. 25, 2016 (EP) 16166916.3

(51) Int. Cl. A61K 9/24; A61K 31/4725; A61K 31/426; A61K 9/20

(52) U.S. Cl. CPC A61K 9/209; A61K 31/4725; A61K 9/2095; A61K 9/2013; A61K 9/2009; A61K 31/426

(57) ABSTRACT

The present invention relates to a pharmaceutical multi layer tablet comprising a controlled release part with mirabegron and an immediate release part wherein the immediate release formulation comprises: solifenacin succinate and a water insoluble diluent in an amount of 50 to 99% w/w relative to the total weight of the immediate release part of the tablet. The invention further relates to the use of said composition as a medicament, particularly in the treatment of urinary incontinence.

### BACKGROUND OF THE PRESENT INVENTION

Mirabegron is chemically described as (R)-2-(2-aminothiazol-4-yl)-4′-[2-[(2-hydroxy-2-phenylethyl) amino] ethyl-acetanilide or 2-amino-N-[4-[2-[[(2R)-2-hydroxy-2-phenylethyl] amino] ethyl] phenyl]-4-thiazoleacetamide. It has the structure of formula (I).

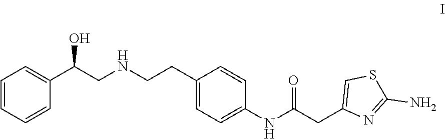 [SMILES: Nc1nc(CC(=O)Nc2ccc(CCNC[C@H](O)c3ccccc3)cc2)cs1]

Mirabegron is an orally active beta-3 adrenoreceptor agonist registered for the treatment of urinary overactive bladder by Astellas Pharma. U.S. Pat. No. 6,346,532 B1 discloses mirabegron or a salt thereof and the process for its preparation.

A mirabegron containing pharmaceutical product is approved under the brand name Betmiga® in the EU and Mirbetriq® in the US as modified release tablets comprising 25 and 50 mg of mirabegron.

Mirabegron is considered to be a Class III compound according to the Biopharmaceutical Classification System (BCS). That means that it has high solubility and low permeability. Based on the assessment report of Betmiga® published by the European Medicines Agency, mirabegron is soluble in water between neutral to acidic pH.

It is known that the bioavailability of mirabegron is affected by the presence of food in the GI tract. Therefore to prevent this food effect, the commercially available pharmaceutical formulations of mirabegron are in the form of a modified-release (MR) tablet formulation based on an orally controlled absorption system (OCAS®) tablet formulation.

The OCAS® system is described in WO9406414 (A1). WO9406414 (A1) describes a hydrogel-type sustained-release preparation comprising (1) at least one drug (tamsulosine as one of the examples), (2) an additive which insures a penetration of water into the core of the preparation and (3) a hydrogel-forming polymer, wherein said preparation is capable of undergoing substantially complete gelation during its stay in the upper digestive tract including stomach and small intestine and is capable of releasing the drug in the lower digestive tract including colon.

The application of the OCAS® system to Mirabegron is described in WO2010038690 (A1). It specifically describes a tablet formulation comprising mirabegron or a pharmaceutically acceptable salt thereof, an additive which ensures penetration of water into the pharmaceutical composition, and a polymer which forms a hydrogel.

Solifenacin is chemically described as 1-azabicyclo[2.2.2]oct-3-yl (1R)-1-phenyl-3,4-dihydro-1H-isoquinoline-2-carboxylate. It has the structure of formula (I).

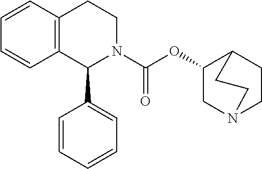 [SMILES: O=C(O[C@H]1CN2CCC1CC2)N1CCc2ccccc2[C@@H]1c1ccccc1]

Solifenacin is a competitive muscarinic acetylcholine receptor antagonist belonging to the class of urinary antispasmodic. Solifenacin is marketed under the tradename Vesicare® as a film-coated tablet that contains either 5 mg or 10 mg solifenacin succinate. The tablet is approved for the symptomatic treatment of urge incontinence and/or increased urinary frequency and urgency as may occur in patients with overactive bladder syndrome.

WO2004047838 teaches that antimuscarinic agents can be combined with β3adrenoreceptors for treating overactive bladder. Both solifenacin and mirabegron are specifically disclosed as examples of antimuscarinic agents and β3adrenoreceptors.

The combination of mirabegron and solifenacin to treat overactive bladder is disclosed in WO2009057685.

A tablet relating to a pharmaceutical composition combining a modified release portion comprising mirabegron with an immediate release portion comprising solifenacin for alleviation of urinary urgency has been disclosed in WO2014034860. This application teaches that such combination of mirabegron and solifenacin interferes with the release rate of solifenacin and that this problem is resolved by adding calcium stereate to the solifenacin formulation.

Further, WO2015129893 teaches that a combination formulation comprising a controlled release portion comprising mirabegron and an immediate release part comprising solifenacin, presents stability problems because of the formation of impurities in the mirabegron controlled release part. These impurities modify the dissolution rate of mirabegron. The application teaches that this problem is solved by incorporating a water-soluble macromolecule or crystalline sugars in the solifenacin immediate release part of the formulation.

Thus, in view of the prior art cited above, there is still a need for alternative pharmaceutical compositions comprising mirabegron and solifenacin which are stable and suitable for use on a commercial scale.

### BRIEF DESCRIPTION OF THE PRESENT INVENTION

The present invention provides a multi layer tablet composition comprising:
A controlled release part comprising mirabegron; and
An immediate release part comprising:
   a) Solifenacin succinate;
   b) A water insoluble diluent, in an amount of 50 to 99% w/w relative to the total weight of the immediate release part of the tablet.

Additionally, the invention provides a process for preparing said tablet composition.

Said pharmaceutical composition may be used as a medicament, particularly in the treatment of urinary incontinence.

### BRIEF DESCRIPTION OF THE DRAWINGS

FIG. 1 depicts the process for making the immediate release part of examples 1 and 4.

FIG. 2 depicts the process for making the immediate release part of examples 2, 3, 5, and 6.

FIG. 3 depicts the process for making the immediate release part of example 7.

FIG. 4 depicts the process for making the controlled release part of example 8.

FIG. 5 depicts the process for making the controlled release part of example 9

FIG. 6 depicts the process for making the controlled release part of example 10.

### DETAILED DESCRIPTION OF THE PRESENT INVENTION

The present invention provides a multi layer tablet composition comprising:
A controlled release part comprising mirabegron; and
An immediate release part comprising:
a) Solifenacin succinate;
b) A water insoluble diluent, in an amount of 50 to 99% w/w relative to the total weight of the immediate release part of the tablet.

A multilayer tablet is typically produced by compactation of different granules in the form of various layers in a single tablet. It generally consists in parallel distinct layers with two or more APIs optionally along with functional or non functional placebo layers, sometimes to avoid interaction between different incompatible layers. More preferably the tablet of the invention is a bilayer tablet. Bilayer tablets are suitable for sequential and simultaneous release of two different APIs. In that case, one layer is immediate release and another layer is controlled release. A bilayer tablet according to the present invention contains a controlled release part comprising 5 to 100 mg of mirabegron and an immediate release part comprising 1 to 30 mg of solifenacin and a diluent which is water insoluble. The preferred dosage strengths of mirabegron are 25 and 50 mg; the preferred dosage strengths of solifenacin are 5 and 10 mg. Presently preferred forms are bilayer tablets comprising 25/5 mg, 25/10 mg, 50/5 mg and 50/10 mg of mirabegron and solifenacin respectively.

Each layer comprises different excipients, so as to give suitable properties for compression, lubrication, binding as is well known to one skilled in the art.

In a tablet comprising two APIs, it is not unusual that the two APIs interact with each other or with the excipients of one or the other layer. This can result in stability problems. It is known that a bilayer tablet of mirabegron and solifenacin can give stability problems to mirabegron.

In application WO2015129893, crystalline sugars and water soluble molecules were used as excipients in the solifenacin immediate release part of the bilayer tablet for increasing the stability of mirabegron.

Surprisingly, the inventors of the present invention have found that a formulation containing one or more water insoluble diluent(s) in an amount of 50 to 99% w/w relative to the total weight of the immediate release part of the tablet has a stabilization effect in mirabegron formulation safeguarding the dissolution properties of the mirabegron controlled release portion during the stability period and improves the stability of the solifenacin immediate release portion.

Therefore, a tablet according to the present invention contains an immediate release tablet layer comprising solifenacin and a diluent which is water insoluble.

Diluents are fillers which are used to increase the bulk volume of a tablet or capsule. By combining a diluent with the active pharmaceutical ingredient, the final product is given adequate weight and size to assist in production and handling.

The term “water insoluble” as used herein means that the solubility in water of the diluent is lower than 0.05 g/100 ml water, measured at 20° C. at 1 atm pressure.

In order to get the stabilization effect, the amount of diluent(s) ranges from 50 to 99% w/w relative to the total weight of the immediate release part of the tablet, preferably from 60 to 98% w/w, even more preferably from 70 to 97% w/w relative to the total weight of the immediate release part of the tablet.

Suitable examples of diluents to be used in accordance with the present invention include alkali metal inorganic salts, more preferably calcium or magnesium inorganic salts, such as calcium carbonate, magnesium carbonate or dibasic calcium phosphate. In a preferred embodiment dibasic calcium phosphate is used as a diluent. Dibasic calcium phosphate can be used in its anhydrous form or as a hydrate. Dibasic calcium phosphate has good compactation properties and good flow properties of the coarse grade material.

Another suitable example of diluent to be used in accordance with the invention is microcrystalline cellulose (MCC).

In a most preferred embodiment microcrystalline cellulose and dibasic calcium phosphate are used together as a diluent, for instance as in the commercial available Avicel DG which is a powder containing 75% of microcrystalline cellulose and 25% anhydrous dibasic calcium phosphate. When MCC and/or phosphate derivatives such as dicalcium phosphate are used, the stability of the bilayer tablet of the invention is improved.

The tablet composition of the present invention preferably contains at least one glidant in the immediate release part with a specific surface area≤400 m2/g.

A glidant is added in order to improve the flowability of the immediate release part of the multilayer tablet. Suitable glidants are magnesium stereate, magnesium silicate, starch, talc and colloidal silicon dioxide. Colloidal silicon dioxide is the most preferred glidant because it reduces van der Waals attractive forces between excipients and solifenacin resulting in an enhanced flowability. It also enhances the uniformity content of solifenacin in powder blend. A range from 0.05 to 10% w/w of glidant is preferred relative to the total weight of immediate release part of the tablet, more preferred is a range from 0.1 and 5% w/w, even more preferred is a range from 0.2 and 2% w/w relative to the total weight of immediate release part of the tablet.

In order to avoid interactions with the release of mirabegron, a rapid disintegration time of the immediate release part of the tablet is desired. To achieve this goal disintegrant can be added to the formulation.

Disintegrants are agents added to tablet formulations to promote the breakup of the tablet (and capsule “slugs’) into smaller fragments in an aqueous environment thereby increasing the available surface area and promoting a more rapid release of the drug substance.

Suitable examples of disintegrants to be used in accordance with the present invention include crospovidone, starch, sodium croscarmellose and mixtures thereof. In a preferred embodiment sodium croscarmellose is used as a disintegrant to avoid oxidation impurities. A range from 1 to 10% w/w of disintegrants is preferred, more preferred is a range from 2 to 5% relative to the total weight of the immediate release part of the tablet.

Optionally other excipients can be used in the tablet of the present invention. These excipients can be chosen from binders and lubricants.

Binders ensure that tablets and granules can be formed having the desired or required mechanical strength, and they give volume to low active dose tablets. Binders which are suitable for use in accordance with the present invention include povidone, hydroxypropyl methylcellulose, hydroxy propylcellulose, and sodium carboxyl methylcellulose.

The tablet composition of the invention may also contain a lubricant. Lubricants are generally used in order to reduce sliding friction. In particular, to decrease friction at the interface between a tablet's surface and the die wall during ejection, and reduce wear on punches and dies. Suitable lubricants to be used in accordance with the present invention include magnesium stearate, calcium stearate, stearic acid, glyceryl behenate, hydrogenated vegetable oil, and glycerine fumarate. Preferably, magnesium stereate is used as lubricant. A preferred multilayer tablet is a tablet wherein the immediate release part has:
a. A water insoluble diluent in an amount of 50 to 99% w/w relative to the total weight of the immediate release part of the tablet, preferably of 60 to 98%, even more preferably of 70 to 97% w/w relative to the total weight of the immediate release part of the tablet.
b. A glidant with a specific surface area≤400 m2/g in an amount 0.05 to 10% w/w relative to the weight of the immediate release part of the tablet.
c. A disintegrant in an amount 1 to 10% w/w relative to the total weight of the immediate release part of the tablet.

The bilayer tablet of the present invention is stable and shows an in vitro dissolution profile wherein mirabegron is released at least 10 to 50% within 3 hours, at least 30 to 70% within 5 hours and at least 60%, preferably 80%, within 10 hours when the composition is subjected to a dissolution study in 900 ml phosphate buffer (pH 6.8) using a USP apparatus 1 (basket) at 100 rpm at 37° C.

The term “stable” as used herein means that tablets comply with the purity and the dissolution specification when subjected to a 6 months stability study at the accelerated stability conditions of 40° C. and 75% RH.

In a preferred embodiment of the invention the immediate release part of the tablet has a dissolution rate of 70% or more in 10 min, even more preferred 80% or more in 10 minutes, when the composition is subjected to a dissolution study in 900 ml water using a USP apparatus 2 (paddles) at 50 rpm at 37° C.

The pharmaceutical compositions described herein can be made using conventional methods and equipment well-known in the art such as wet granulation, dry granulation or mixing. Wet granulation is preferred for the immediate release part of the multilayer tablet.

The controlled release part of the tablet can be made using conventional methods and equipment well-known in the art such as wet granulation, dry granulation or mixing.

The present invention further relates to a tablet composition as described hereinabove, wherein the immediate release part is prepared by a wet granulation process, which process comprises the steps of:
a. Combining solifenacin with a water insoluble diluent;
b. Wet granulating the resulting mixture with a granulation solvent;
c. Drying the resulting granulate;
d. Further mixing the obtained granulate with one or more pharmaceutically acceptable excipients to form a further mixture.

Solvents suitable to perform the wet granulation are water or alcohols. Alcohols are preferred. When alcohols are used the drying process is faster. Furthermore, when alcohol is used the drug product contains less impurities. The preferred alcohol is ethanol. Most preferred alcohol is ethanol 96%.

The controlled release part of the tablet can be made using conventional methods and equipment well-known in the art such as wet granulation, dry granulation or mixing.

The milling speed of the modified release layer may affect the granulate particle size and consequently its compressibility and compactability. If an impact milling machine is used a milling speed between 1000-1500 rpm is recommended in order obtain good flowability and compactability properties. On the contrary, if a screening milling machine is used it is preferred to work at a milling speed between 150-450 rpm.

In the multilayer tablets the interaction of the different APIs can result in stability problems. In order to avoid these problems, the particles of mirabegron and/or solifenacin may be optionally coated. Another option is to coat the granules of solifenacin and/or mirabegron obtained in the above processes. Furthermore, a non functional layer (layer without API) can be added between the mirabegron and solifenacin layer to avoid the interactions of the two drugs. This non functional layer can comprise excipients such as sugars, for instance lactose, cellulose derivatives such as MCC or phosphates derivatives such as dicalcium phosphate.

The apparatus and method used in the step of forming the multilayer tablet are well known in the art. Bi-layered tablets may be prepared by laminating the modified release portion and the immediate release portion, or by compressing the modified release portion and subsequently compressing the immediate release portion; a method of preparing multilayered tablets by adding a drug-free layer between the modified release portion and the immediate release portion.

Examples of a tabletting machine include a multilayered rotary tabletting machine.

The multilayer tablet may be further coated with a film coat.

The tablet composition of the present invention is suitable for use as a medicament e.g for the treatment of urinary incontinence.

The following examples are intended to illustrate the scope of the present invention but not to limit it thereto.

### Preparation of the Immediate Release Part of the Multilayer Tablet

### Examples 1, 2 and 3

TABLE 1
Qualitative and quantitative formula examples 1, 2, 3

| | | | Example 2 | | Example 3 | |
|---|---|---|---|---|---|---|
| | Example 1 | | Wet granulation | | Wet granulation | |
| | Dry process | | (Water) | | (Alcohol) | |
| Component/Function | mg | % | mg | % | mg | % |
| Solifenacin succinate/API | 5.00 | 3.0% | 5.00 | 3.0% | 5.00 | 3.0% |
| Calcium phosphate | 156.94 | 94.2% | 156.94 | 94.2% | 152.24 | 91.4% |
| Dibasic Anydrous | | | | | | |
| (DICAFOS)/Diluent | | | | | | |
| Crospovidone/Disintegrant | 2.00 | 1.2% | 2.00 | 1.2% | 6.70 | 4.0% |
| Colloidal silicon dioxide | 1.00 | 0.6% | 1.00 | 0.6% | 1.00 | 0.6% |
| (Aerosil)/Glidant | | | | | | |
| Magnesium | 1.66 | 1.0% | 1.66 | 1.0% | 1.66 | 1.0% |
| stearate/Lubricant | | | | | | |
| Alcohol absolute | — | — | — | — | 0.0072 mL | |
| Purified Water | — | — | 0.0140 mL | | — | — |
| Total weight content | 166.60 | 100.0% | 166.60 | 100.0% | 166.60 | 100.0% |

### Examples 4, 5 and 6

TABLE 2
Qualitative and quantitative formula example 4, 5, 6

| | | | Example 5 | | Example 6 | |
|---|---|---|---|---|---|---|
| | Example 4 | | Wet granulation | | Wet granulation | |
| | Dry process | | (Water) | | (Alcohol) | |
| Component/Function | mg | % | mg | % | mg | % |
| Solifenacin succinate/API | 5.00 | 3.0% | 5.00 | 3.0% | 5.00 | 3.0% |
| Microcrystalline cellulose: | 156.94 | 94.2% | 156.94 | 94.2% | 156.94 | 94.2% |
| Dibasic Calcium Phosphate | | | | | | |
| Dihydrate (Avicel | | | | | | |
| DG)/Diluent | | | | | | |
| Crospovidone/Disintegrant | 2.00 | 1.2% | 2.00 | 1.2% | 2.00 | 1.2% |
| Aerosil/Glidant | 1.00 | 0.6% | 1.00 | 0.6% | 1.00 | 0.6% |
| Magnesium | 1.66 | 1.0% | 1.66 | 1.0% | 1.66 | 1.0% |
| stearate/Lubricant | | | | | | |
| Alcohol absolute | — | — | — | — | 0.0278 mL | |
| Purified Water | — | — | 0.0610 mL | — | — | — |
| Total weight content | 166.60 | 100.0% | 166.60 | 100.0% | 166.60 | 100.0% |

### Example 7 Using Wet Granulation

| | Example 7 | |
|---|---|---|
| | Wet granulation | |
| Component/Function | mg | % |
| Solifenacin succinate/API | 5.00 | 3.0% |
| 75% MCC:25% anhydrous dibasic calcium | 153.82 | 92.33% |
| phosphate (Avicel DG) | | |
| Colloidal silicon dioxide (Aerosil 200 VV) | 1.00 | 0.60% |
| Sodium croscarmellose | 5.00 | 3.00% |
| Magnesium stearate | 1.66 | 1.00% |
| Red iron oxide | 0.12 | 0.07% |
| Ethanol 96% | 31.77 | |
| Final weight | 166.60 | 100.0% |

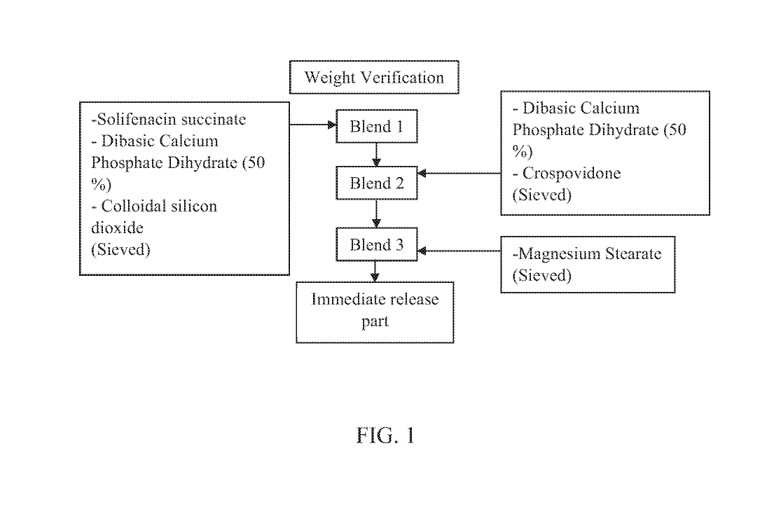

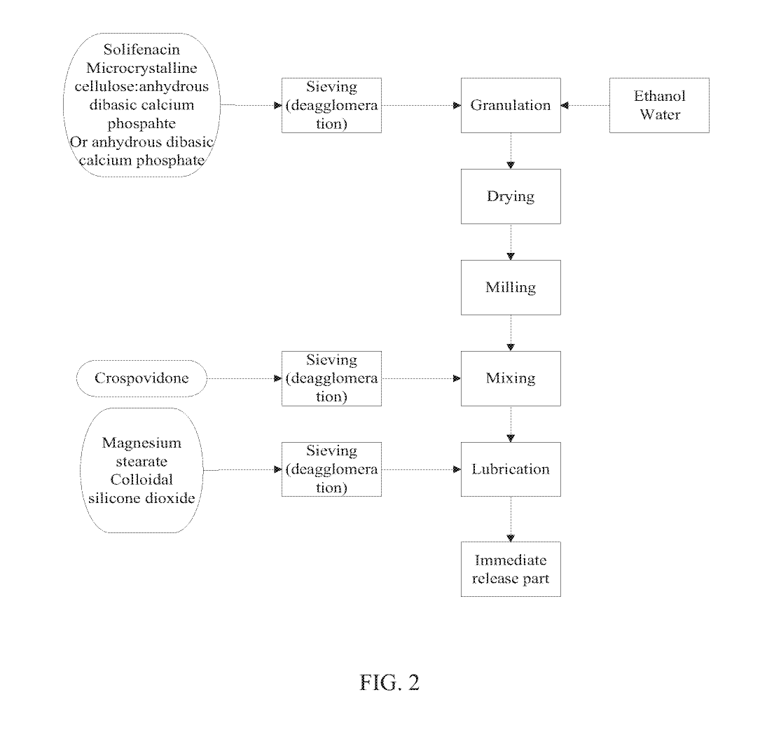

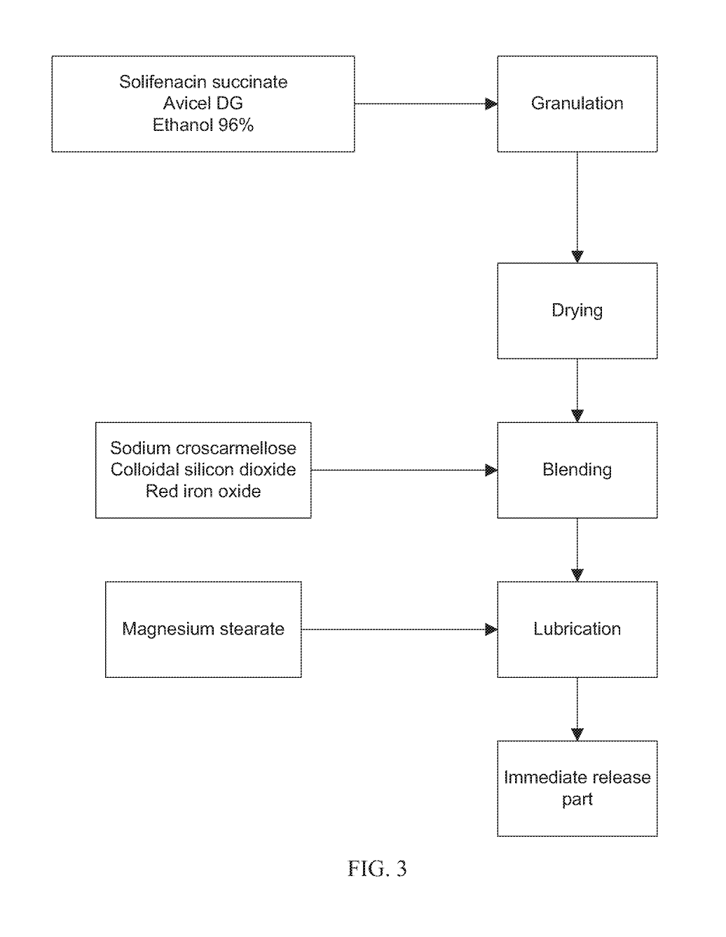

The immediate release part of examples 1 and 4 was made according to the process depicted in FIG. 1. The immediate release part of Examples 2, 3, 5 and 6 was made according to the process depicted in FIG. 2. The immediate release part of Example 7 was made according to the process depicted in FIG. 3.

### Preparation of the Controlled Release Part of the Multilayer Tablet

### Example 8

TABLE 3
Qualitative and quantitative formula example 8

| | | |
|---|---|---|
| Components | mg/tablet | % |

Intragranular

| | | |
|---|---|---|
| Mirabegron | 50.000 | 20.0 |
| PEO 2,000,000 (Polyox | 35.000 | 14.0 |
| WSR N60K) | | |
| Microcrystalline cellulose | 63.750 | 25.5 |
| (Vivapur 101) | | |

Extragranular

| | | |
|---|---|---|
| PEO 900,000 (Polyox WSR | 35.000 | 14.0 |
| 1105) | | |
| Microcrystalline cellulose | 63.750 | 25.5 |
| (Vivapur 102) | | |
| Magnesium stearate | 2.500 | 1.0 |
| Ethanol:water (85:15) | qs | qs |

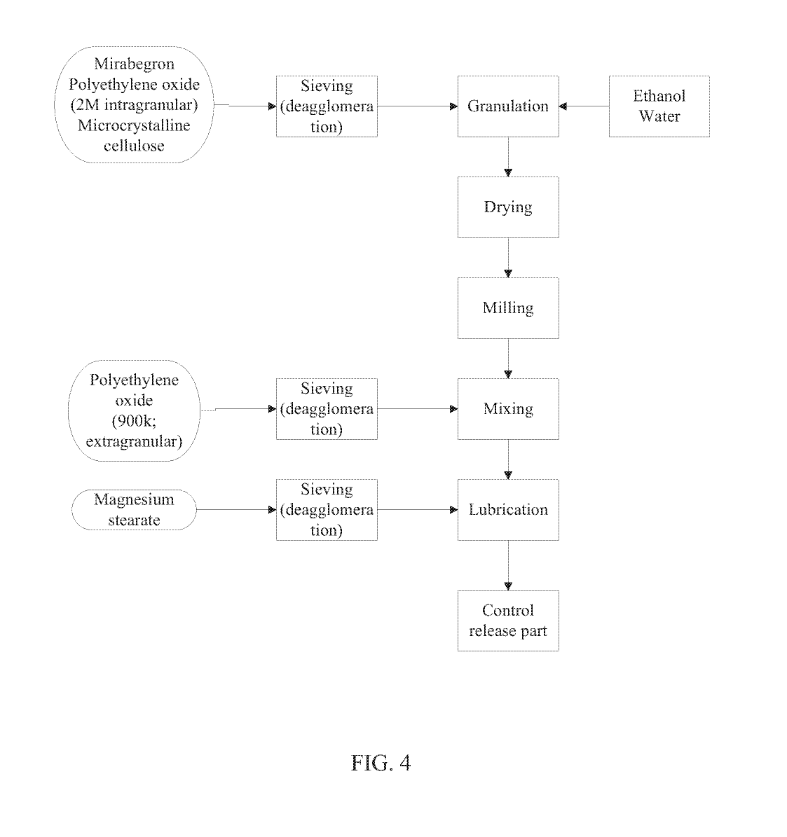

The controlled release part of Example 8 was made according to the process depicted in FIG. 4.

### Example 9

TABLE 4
Qualitative and quantitative formula example 9

| | | |
|---|---|---|
| Components | mg/tablet | % |

Intragranular

| | | |
|---|---|---|
| Mirabegron | 50.000 | 20 |
| PEO 7,000,000 (PEO-20NF) | 25.400 | 10.16 |
| Polyethylene glycol 8,000 P | 164.600 | 65.84 |
| Hydroxypropyl cellulose | 7.500 | 3 |
| (Klucel EXF) | | |
| Magnesium stearate | 1.250 | 0.5 |

Extragranular

| | | |
|---|---|---|
| Magnesium stearate | 1.250 | 0.5 |

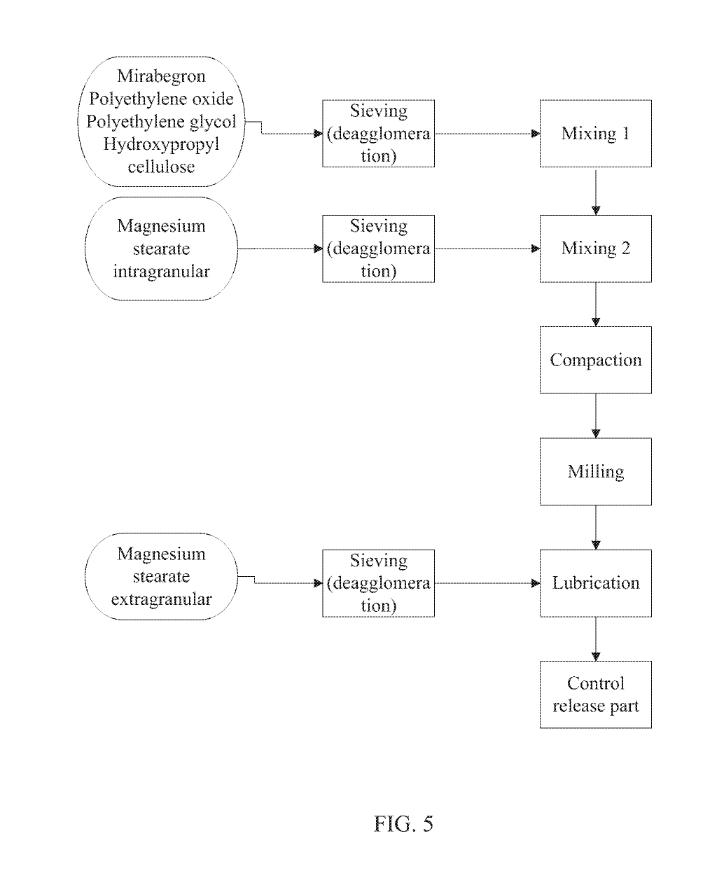

The controlled release part of Example 9 was made according to the process depicted in FIG. 5.

### Example 10

TABLE 5
Qualitative and quantitative formula example 10

| | | |
|---|---|---|
| Components | mg/tablet | % |

Intragranular

| | | |
|---|---|---|
| Mirabegron | 25.000 | 20.1 |
| PEO 2,000,000 (Polyox | 35.000 | 28.2 |
| WSR N60K) | | |
| Hydroxypropyl cellulose | 3.750 | 3.0 |
| (Klucel EXF) | | |

Extragranular

| | | |
|---|---|---|
| PEG8000 (Polyglycol | 59.800 | 48.2 |
| 8000P) | | |
| Magnesium stearate | 0.625 | 0.5 |
| BHT | 0.2 | 0.161063 |

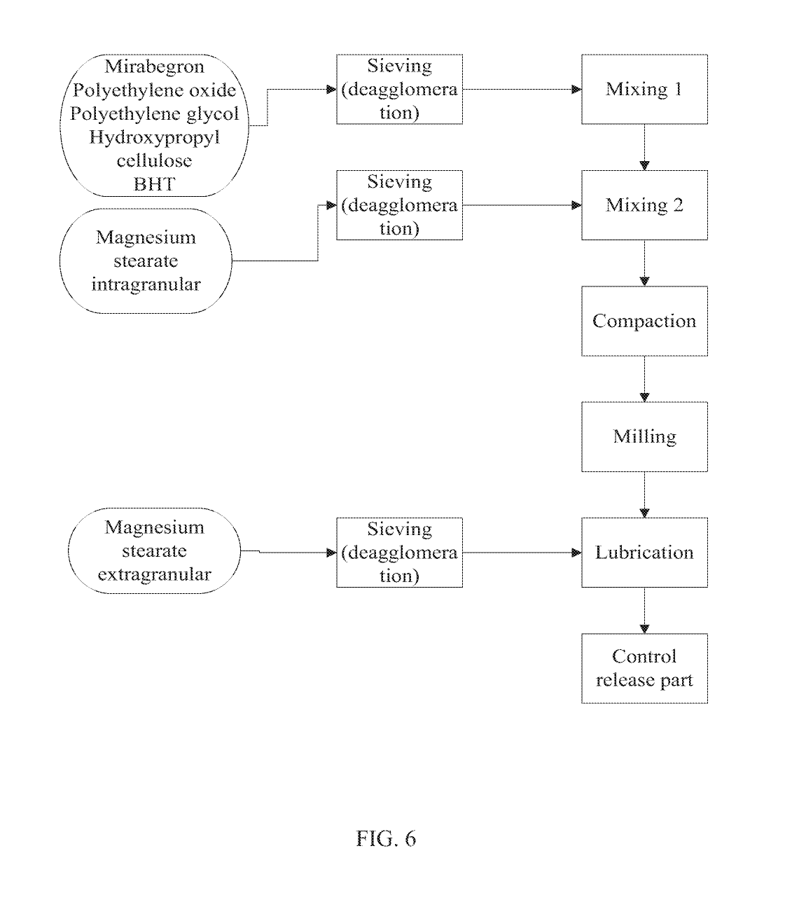

The controlled release part of Example 10 was made according to the process depicted in FIG. 6.

Tabletting the Multilayer Tablet

Using a multilayered rotary tabletting machine, one part of the modified release portion (from examples 8-10) and one part of the immediate release portion (from examples 1-5) were formed into bilayered tablets, to obtain a pharmaceutical composition for oral administration of the present invention containing 50 or 25 mg of mirabegron and 5 mg of solifenacin succinate.

Optionally a non functional layer of dicalcium phosphate anhydrous can be added between the mirabegron layer and the solifenacin layer to avoid interaction of the two APIs. For this purpose, mirabegron is compressed, subsequently the dicalcium phosphate layer is compressed and finally the solifenacin layer is added.

1. A multi layer tablet comprising:
i) a controlled release part comprising mirabegron; and
ii) an immediate release part comprising:
a) solifenacin succinate; and
b) a water insoluble diluent in an amount of 50 to 99% w/w relative to the total weight of the immediate release part of the tablet.

2. The tablet according to claim 1, wherein the diluent is an alkali earth metal inorganic salt.

3. The tablet according to claim 2, wherein the diluent is a calcium or magnesium inorganic salt.

4. The tablet according to claim 1, wherein the diluent is dicalcium phosphate or microcrystalline cellulose or a combination of both.

5. The tablet according to claim 1, further comprising a glidant in the immediate release part with a specific surface area≤400 m²/g in an amount which is 0.05 to 10% w/w relative to the weight of the immediate release part of the tablet.

6. The tablet according to claim 5, wherein the glidant is silicone dioxide

7. The tablet according to claim 5, further comprising a disintegrant.

8. The tablet according to claim 7, where the disintegrant is sodium croscarmellose.

9. The tablet according to claim 1, wherein the solifenacin immediate release part has:
a. a water insoluble diluent in an amount of 50 to 99% w/w relative to the total weight of the immediate release part of the tablet;
b. a glidant with a specific surface area≤400 m²/g in an amount of 0.05 to 10% w/w relative to the weight of the immediate release part of the tablet; and
c. a disintegrant in an amount of 1 to 10% w/w relative to the total weight of the immediate release part of the tablet.

10. The tablet according to claim 1, wherein the dissolution rate of the immediate release part is 70% or more in 10 min.

11. A process for the preparation of the immediate release part of the multilayer tablet according to claim 1, comprising:
a. Combining solifenacin with a water insoluble diluent;
b. Wet granulating the resulting mixture with a granulation solvent;
c. Drying the resulting granulate;
d. Further mixing the obtained granulate with one or more further pharmaceutically acceptable excipients to form a further mixture.

12. The process of claim 11 wherein alcohol is used as a granulation solvent.

13. The process of claim 12 where ethanol is used as a granulation solvent.

14. A method of treating urinary incontinence in a patient, which comprises administering to said patient the tablet according to claim 1.
